(19) United States
(12) Patent Application Publication
(10) Pub. No.: US 2011/0250250 A1
(43) Pub. Date: Oct. 13, 2011

(54) COMPOSITIONS AND METHODS FOR SPF ENHANCEMENT BY HIGH CONCENTRATION COSMETIC POWDER FORMULATIONS

(75) Inventors: SHIGERU KISHIDA, Storrs, CT (US); YOSHI KAWASAKI, Woodstock, CT (US); RONALD YAKUPCIN, Matamoras, PA (US)

(73) Assignee: U.S. COSMETICS CORPORATION, Dayvill, CT (US)

(21) Appl. No.: 13/084,242

(22) Filed: Apr. 11, 2011

Related U.S. Application Data
(60) Provisional application No. 61/323,678, filed on Apr. 13, 2010.

(51) Int. Cl. A61K 8/92; A61K 8/97; A61K 8/02; A61Q 17/04

(52) U.S. Cl. 424/401; 424/59; 424/60

(57) ABSTRACT

The present disclosure relates generally to cosmetic formulations wherein cosmetic powder materials potentiate the UV protection factor provided by an organic sunscreen. The present disclosure relates particularly, but not by way of limitation, to UV-protecting cosmetic formulations comprising cosmetic powders and having low loadings of organic sunscreens.

The present application claims the priority of U.S. Provisional Application 61/323,678, filed Apr. 13, 2010, the entire contents of which is hereby incorporated by reference for all purposes.

### FIELD OF THE INVENTION

The present disclosure relates generally to cosmetic formulations wherein cosmetic powder materials potentiate the UV protection factor provided by an organic sunscreen. The present disclosure relates particularly, but not by way of limitation, to UV-protecting cosmetic formulations comprising cosmetic powders and having low loadings of organic sunscreens.

### BACKGROUND

Many lotion-type sunscreens are currently available on the market. Typically, sunscreen formulas are water-in-oil (W/O) or oil-in-water (O/W) emulsions or are anhydrous systems. In order to obtain a high sun protection factor (SPF) and particularly a high protection factor relative to UV-A radiation (PFA), sunscreen formulations typically incorporate extensive amounts of oil-based, UV-active materials. The use of large amounts of oil-based, UV-actives causes the texture of the resulting sunscreens to be oily, greasy, tacky, and somewhat opaque. Oils are also undesirable because they may enhance the transdermal permeation of other formulation ingredients including ingredients for which transdermal administration may be inappropriate. In addition to these undesirable properties, the high loading of oil-based UV-actives often causes adverse skin reactions in sensitive individuals.

Commercial sunscreens are typically formulated to yield about 1 to 2 SPF units per weight percent (wt %) UV-active ingredient. For example, typical SPF 20 sunscreen formulations contain approximately 13% UV-active materials. It is often desirable to formulate sunscreen with much higher SPF ratings. To formulate sunscreens at the higher SPF rating requires corresponding increases in the concentration of oil-based, organic LTV-actives.

It is desirable to formulate sunscreens with increasingly high SPF values to confer higher levels of protection. However, the current formulation metric implies higher degrees of unwanted side-effects. If organic UV absorbers are used in formulations at the lowest possible level, tactile issues and safety concerns would be ameliorated. Also, production costs would be lower as lesser amounts of raw materials are used. Therefore, there exists a need to formulate sunscreens having lower amounts of organic UV-actives.

Other objects and advantages will become apparent from the following disclosure.

### SUMMARY OF INVENTION

The present disclosure relates to a cosmetic formulation comprising at least one organic, UV-active material and at least one cosmetic powder material such that the formulation has an SPF Index of at least 3.0. According to aspects of the disclosure, the cosmetic formulation has an SPF Index of at least 4.0. According to aspects of the disclosure, the cosmetic formulation has an SPF Index of at least 6.0. According to aspects of the disclosure, the cosmetic formulation has an SPF Index of at least 8.0. According to aspects of the disclosure, the cosmetic formulation has an SPF Index of at least 10.0.

The present disclosure relates to an SPF-boosting composition comprising less than about 25 wt % of a non-volatile oil, at least 40 wt % of an aqueous phase, and about an SPF-inflection concentration of a cosmetic powder. According to aspects of the disclosure, the non-volatile oil comprises a UV-protective agent. According to aspects of the disclosure, the non-volatile oil may further comprise a general cosmetic emollient with a relatively high boiling point.

According to aspects of the disclosure, the cosmetic formulation comprises at least one organic, UV-active. According to aspects of the disclosure, the organic UV-active is any organic sunscreen which absorbs, blocks, or otherwise mitigates ultraviolet radiation.

According to an aspect of the disclosure, the invention excludes volatile silicone oils.

According to aspects of the disclosure, the cosmetic formulation comprises a cosmetic powder material present at a concentration that potentiates the UV-blocking activity of an organic UV-active material. According to an aspect of the disclosure, the cosmetic powder material is present in at least an inflection concentration.

According to an aspect of the disclosure, the cosmetic powder materials may be modified by having an organic material bonded onto a surface thereof.

According to an aspect of the disclosure, the surfaces of the cosmetic powder materials may be un-modified.

Still other aspects and advantages of the present invention will become readily apparent by those skilled in the art from the following detailed description, wherein it is shown and described preferred embodiments of the invention, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### BRIEF DESCRIPTION OF DRAWINGS

The invention is best understood from the following detailed description when read in connection with the accompanying drawing. Included in the drawing are the following figures:

FIG. 1 shows the sigmoid dependence of the value of SPF on the concentration of surface-modified cosmetic powder materials;

FIG. 2 shows the sigmoid dependence of the value of in vitro SPF on the concentration of kaolin treated with triethoxycaprylylsilane (and) aluminum dimyristate (and) disodium stearoyl glutamate;

FIG. 3 shows the sigmoid dependence of the value of in vitro PFA on the concentration of kaolin treated with triethoxycaprylylsilane (and) aluminum dimyristate (and) disodium stearoyl glutamate;

FIG. 4 shows the sigmoid dependence of in vitro SPF on the concentration of untreated kaolin; and,

FIG. 5 shows the sigmoid dependence of in vitro PFA on the concentration of untreated kaolin;

It is to be noted, however, that the appended drawings illustrate only typical embodiments of this invention and are therefore not to be considered limiting of its scope, for the invention may admit to other equally effective embodiments.

### DETAILED DESCRIPTION OF A PREFERRED EMBODIMENT

Reference is made to the figures to illustrate selected embodiments and preferred modes of carrying out the invention. It is to be understood that the invention is not hereby limited to those aspects depicted in the figures.

The composition of the present invention comprises up to about 25 wt % of a non-volatile oil. The non-volatile oil comprises an organic, UV-active material that functions as a UV-protective agent (a “sun block”). Preferably, two or more organic, UV-actives are used to provide a wide spectrum of protection in the UV region. For example, a combination of at least one UV protecting agent that mainly provides protection against UVA light, and at least one UV protecting agent that mainly provides protection against UVB light, may be used.

A wide variety of conventional UV protecting agents are suitable for use herein. Non-limiting exemplary organic, UV-actives include: 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene (Parsol 340, DSM), oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, avobenzone (Parsol 1789, DSM), avobenzone, PABA, octyldimethyl-PABA, Phenylbenzimidazole sulfonic acid, Cinoxate, Dioxybenzone (Benzophenone-8), Oxybenzone (Benzophenone-3), Homosalate, Menthyl anthranilate, Octisalate, Sulisobenzone, Trolamine salicylate, Terephthalylidene Dicamphor Sulfonic Acid, 4-Methylbenzylidene camphor, Methylene Bis-Benzotriazolyl Tetramethylbutylphenol, Bis-ethylhexyloxyphenol methoxyphenol triazine, bisimidazylate, Drometrizole Trisiloxane, Octyl triazone, Diethylamino Hydroxybenzoyl Hexyl Benzoate, Iscotrizinol, Polysilicone-15, Amiloxate, Ethylhexyl Dimethoxybenzylidene Dioxoimidazolidine Propionate, and mixtures thereof.

In addition to a UV-active, the non-volatile oil may comprise an ancillary oil which may be a solvent for one or more of the UV-active oils. The ancillary oil may provide desirable cosmetic properties such as emolliency and a good “skin feel.” A preferred, but non-limiting ancillary oil is isopropyl myristate.

Non-volatile cosmetic emollient oils having a relatively high boiling point and function as a skin feel modifiers include, but are not hydrocarbons, fatty alcohols, fatty acids, non-volatile silicone oils, and esters such as glycerides and glycol esters.

Suitable ancillary oils include, but are not limited to isotridecyl isononanoate, isostearyl isostearate, isocetyl isosteatrate, isopropyl isostearate, isodecyl isonoanoate, cetyl octanoate, isononyl isononanoate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, egg yolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice pan oil, glycerol trioctanate, glycerol triisopalmiatate, trimethylolpropane triisostearate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, tridecyl isononanoate with tradename Crodamol TN available from Croda, Hexalan available from Nisshin Seiyu, and tocopherol acetates available from Eisai.

Non-volatile cosmetic emollients may include waxes such as, but not limited to paraffin wax, microcrystalline wax, ozokerite wax, ceresin wax, carnauba wax, candelilla wax, and eicosanyl behenate.

Non-volatile silicon oils may be used including, but not limited to polymethylphenylsiloxane, polydiphenylsiloxane, polydiethylsiloxane, polydimethylsiloxane (dimethicone). For purposes of the present disclosure, a non-volatile silicon oil is defined as one that has a kinematic viscosity greater than 10 centiStokes (cSt).

Suitable ancillary oils include polyalkyl or polyaryl siloxanes as disclosed in U.S. Pat. No. 6,936,241.

Suitable ancillary oils useful herein include the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

The non-volatile oil may not comprise a “volatile” silicone oil. A specifically excluded volatile silicone oil is decamethylcyclopentanasilaxane, commonly known as “D5.”

The term “powder material,” or “cosmetic powder material,” as used herein includes organic and inorganic pigments, pigment extenders, inorganic and organic beads, metals, metal oxide powders, plastics, fillers for plastics, dehydrated dairy products. As known in the art, a powder is any solid, dry material consisting of extremely small, flowable particles.

The powder material may be an inorganic powder, such as an extender pigment, non-limiting examples of extender pigments include: mica, sericite, talc, kaolin, synthetic mica, muscovite, phlogopite, epidolite, biotite, calcium carbonate, magnesium carbonate, calcium phosphate, alumina, magnesium oxide, aluminum hydroxide, barium sulfate, magnesium sulfate, silicic acid, silicic anhydride, magnesium silicate, aluminum silicate, aluminum magnesium silicate, calcium silicate, barium silicate, strontium silicate, silicon carbide, magnesium aluminate, magnesium metasilicate aluminate, chlorohydroxyaluminum, clay, bentonite, zeolite, smectite, hydroxyapatite, ceramic powder, boron nitride and silica.

The powder material may be a special composite extender pigment such as, but not limited to Excel Mica, Excel Pearl and Powder La Vie sold by Miyoshi Kasei, Inc.; white pigments such as titanium dioxide, zinc oxide and cerium oxide; color pigments such as red iron oxide, yellow iron oxide, black iron oxide, chromium oxide, chromium hydroxide, Prussian blue, ultramarine, inorganic blue pigment, carbon black, titanium oxide, mango violet, cobalt violet, laked tar dye and laked natural dye; bright pigments such as bismuth oxychloride, mica titanium, fish scale guanine, a powder obtained by coating synthetic mica with titanium dioxide, a powder obtained by coating silica flakes with titanium dioxide as sold under a trade name “Metashine” by Nippon Sheet Glass Co., Ltd., a powder obtained by coating alumina flakes with tin oxide and titanium dioxide, a powder obtained by coating aluminum flakes with titanium dioxide, a powder obtained by coating copper flakes with silica as sold by Eckart, U.S.A., a powder obtained by coating bronze flakes with silica and a powder obtained by coating aluminum flakes with silica;

The powder material may be an organic powder, non-limiting examples of which include a wool powder, a polyamide powder, a polyester powder, a polyethylene powder, a polypropylene powder, a polystyrene powder, a polyurethane powder, a benzoguanamine powder, a tetrafluoroethylene powder, a polymethyl methacrylate powder, a cellulose powder, a silk powder, a silicone powder, a silicone rubber powder, a styrene acrylic copolymer, a divinylbenzene.styrene copolymer, synthetic resin powders such as a vinyl resin, a urea resin, a phenol resin, a fluoro resin, a silicon resin, an acrylic resin, a melamine resin, an epoxy resin and a polycarbonate resin, a fine crystalline fibrous powder, a starch powder, an acylated lysine powder, a long-chain alkyl phosphate metallic salt powder, or a metal soap powder.

The powder material may be a mixture of any or all of the suitable powder materials.

With respect to the form of the powder used, the form which is ordinarily incorporated in cosmetics, such as a mixture, a composite, or an adhered powder can be used. For example, these powders can be used by being complexed or doped as required. Examples thereof include a powder obtained by coating an inorganic color pigment such as red iron oxide with silicic anhydride, a powder obtained by coating nylon with a white pigment, a powder obtained by coating an extender pigment with a finely divided white pigment and the like.

Suitable powder materials include inorganic pigments such as, but not limited to titanium dioxides, zinc oxides, zirconium dioxides, iron oxides (including yellow, red, and black), ultramarines (such as ultramarine blue, ultramarine violet, etc.), and manganese violet.

The cosmetic powder materials may be coated with a surface-active agent. The term “surface-active agent” encompasses all chemical agents known in the art that have the ability to affect the surface of a compound, including, but not limited to, surfactants, detergents, wetting agents, and emulsifiers. The surface-active agents need not have any special characteristics and may be nonionic or anionic, hydrophobic or hydrophilic.

Preferably, the surface-active agents have one or more reactive groups, such as a carboxyl group, a phosphorous group, a sulfur group, or a silane group. The surface-active agent may or may not contain one or more hydroxyl groups or alkylene oxide moieties, such as ethylene oxide or propylene oxide. More preferably, the surface-active agents are acyl collagens, ether carboxylic acids and their salts, lactates, gluconates, amino acids (such as, but not limited to leucine, phenylalanine, thereonine and serine), acyl amino acids and their salts (such as acylglutamates, acylsarcosinates, acylglycinates, and acylalaninates), fatty acids and their salts, silanes (such as organic silane) or glycerol phosphate esters (such as lecithin). Particularly preferred surface-active agents include triethoxycaprylylsilane, methicone, galacturonic acid, glucarolactone, gallic acid, glucoheptanoic acid, 12-hydroxystearic acid, laurylamidobetaine, stearyl amphoacetate, lauryl amphopropionate, stearyl amphopropionate, polyethylene, sodium myristoyl sarosinate, disodium stearoyl glutamate, isostearyl sebacic acid, and combinations thereof.

In certain embodiments, the particulate substrate of the present invention is coated with a plurality of surface-active agents. Tables II and III disclose a kaolin substrate which is coated with three surface-active agents: triethoxycaprylylsilane, myristic acid, and stearoyl glutamate, where each surface-active agent is linked to the kaolin substrate by a polyvalent metal. Table IV discloses silica, mica, bismuth oxychloride, and aluminum calcium sodium silicate substrates, each coated with the same triple coating of surface-active agents. Any two or more surface-active agents may be combined as a plural coating. Plural coatings, and methods to apply them, are disclosed in co-pending applications Ser. Nos. 12/273,495 and 12/115,901, the entire contents of which are incorporated by reference.

The surface-active agents are chemically immobilized onto the surface of the powder material by the methods known in the art, such as those described in one or more of U.S. Pat. Nos. 5,897,868; 6,482,441; 5,744,126; 7,374,783. Surface-active agents may be chemically immobilized by the methods described in one or more of co-pending U.S. application Ser. No. 11/142,468. Each patent and patent publication is herein incorporated by reference in its entirety. Chemical immobilization differs from adding the surface-active agents to the powder material in that the treated powder material has a uniformly chemically bound reaction product.

The surface-active agents are present in an amount of at least 0.1% by weight, based on the weight of the powder material. Preferably, the surface-active agents are present in an amount ranging from about 1.0 to about 200% by weight; more preferably, from about 1.0 to about 60% by weight; and most preferably, from about 2.0 to about 30% by weight.

The inventors have unexpectedly shown that cosmetic powders potentiate the UV-activity of conventional organic UV-actives. Potentiation means that a given level of UV protection may be achieved at a far lower concentration of organic UV-active than is possible in the absence of cosmetic powder.

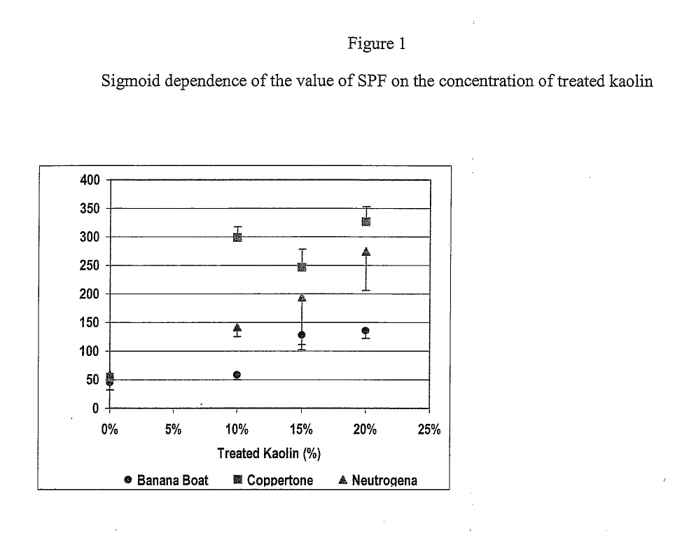

In FIG. 1, the skin protection factor (SPF) yielded by three commercial sunblock formulations is compared in the presence and absence of increasing concentrations of a cosmetic powder. In FIG. 1, the cosmetic powder is kaolin, the surface of which is coated with triethoxycaprylsilane-aluminum dimyristate—disodium stearoyl glutamate—aluminum hydroxide. The coating was applied by methods disclosed in U.S. Pat. Nos. 7,276,113 and 6,482,441 and in U.S. Patent Application Publications 2006/0286048, 2008/0299158, and 2009/0191139. In the absence of cosmetic powder, each of the commercial formulations yields an SPF of approximately 50. The presence of cosmetic powder causes an increase in the observed SPF. Moreover, the observed SPF increases with increasing powder concentrations (each point is plotted as the mean±SD, n=3).

Unexpectedly, the observed SPF does not increase linearly with increasing powder concentrations. Low powder concentrations had little apparent affect on observed SPF values. At high powder concentrations, the process responsible for the increase in SPF appears to saturate at a limiting observed value. Each of the three commercial formulations appeared to give a unique saturation, or “plateau,” value. At intermediate concentrations, the increase in observed SPF appears to follow sigmoid kinetics with an apparent inflection concentration which appears to be unique to each formulation (▪, 7.5%; ▴, off scale; , 12%). Neither a plateau value, nor an inflection in the process, is predictable from the prior art.

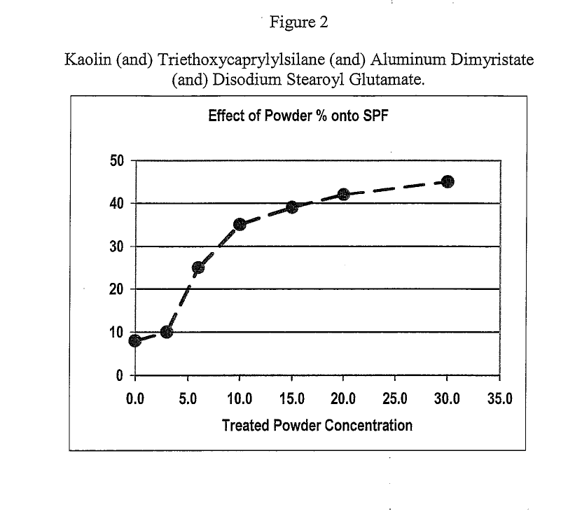

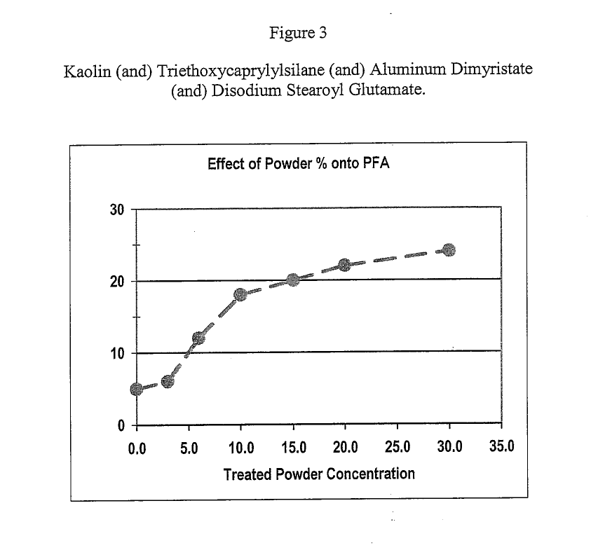

The sigmoid response of SPF (or PFA) to increasing concentrations of surface-treated powders is more clearly shown in Tables II, III, and IV, and FIGS. 2 and 3. In Tables II and III, an oil-in-water emulsion system is formulated with increasing amounts of kaolin the surface of which is treated with triethoxycaprylylsilane (and) aluminum myristate (and) disodium stearoyl glutamate. The data show that SPF (or PFA) increases with increasing powder concentration. Moreover, the increase in sun protection is not a linear function of powder concentration. A sigmoid curve is obtained where SPF (FIG. 2) or PFA (FIG. 3) is plotted as a function of powder concentration.

Table IV shows that the sigmoid response of SPF (or PFA) to increasing concentrations of surface-treated powders does not depend on the presence of a particular substrate. The SPF response to coated powders is demonstrated for silica, bismuth oxychloride, mica, and aluminum calcium sodium silicate substrates. Moreover, a plural coating is not required as Table IV demonstrates for talc coated with methicone or dimethicone and for kaolin coated with triethoxycaprylylsilane.

The ability of surface treated powders to enhance the SPF of a given oil-in-water (O/W) emulsion is not limited to a particular substrate or to a particular surface-treatment. Table IV demonstrates SPF enhancement where the substrate powder is silica, bismuth oxychloride, mica, aluminum calcium sodium silicate, talc, or kaolin powders, coated with any of triethoxycaprylylsilane, methicone, dimethicone, or triethoxycaprylylsilane (and) aluminum myristate (and) disodium stearoyl glutamate.

The powder-dependent, SPF-enhancement effect is not limited to O/W systems. Table V demonstrates powder-dependent SPF enhancement for a water-in-oil (W/O) system.

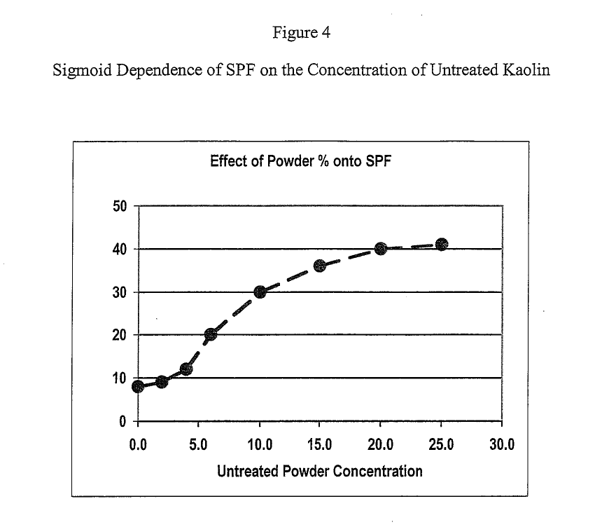

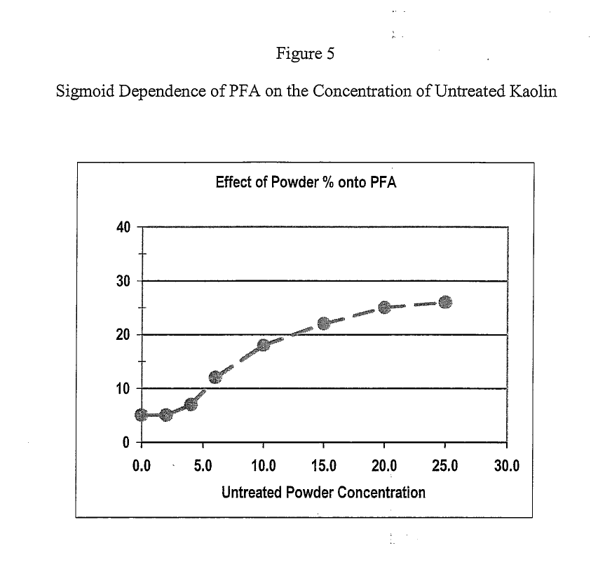

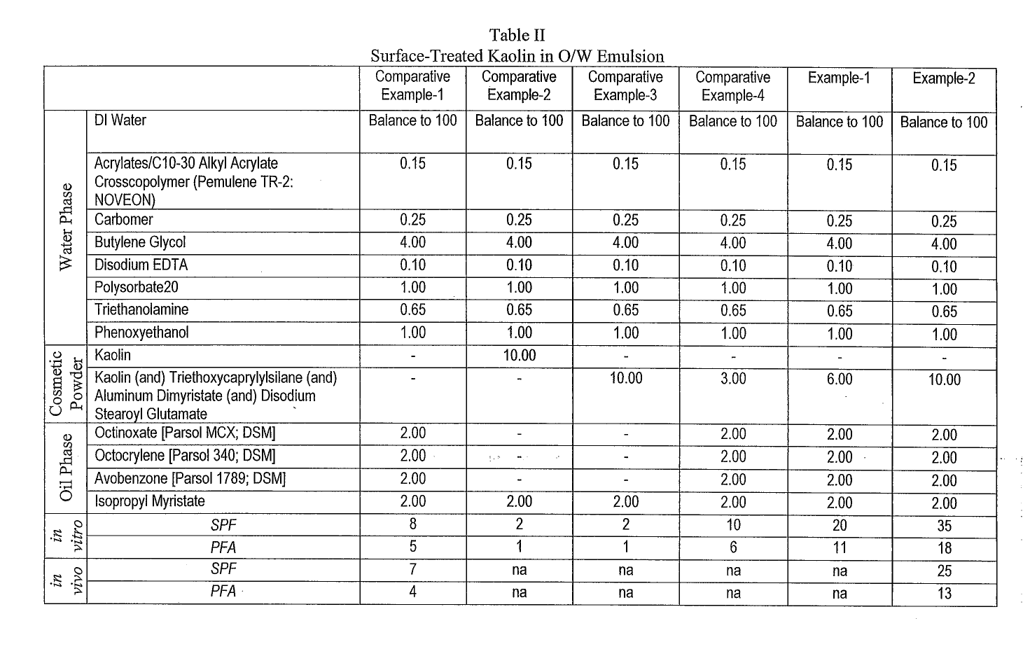

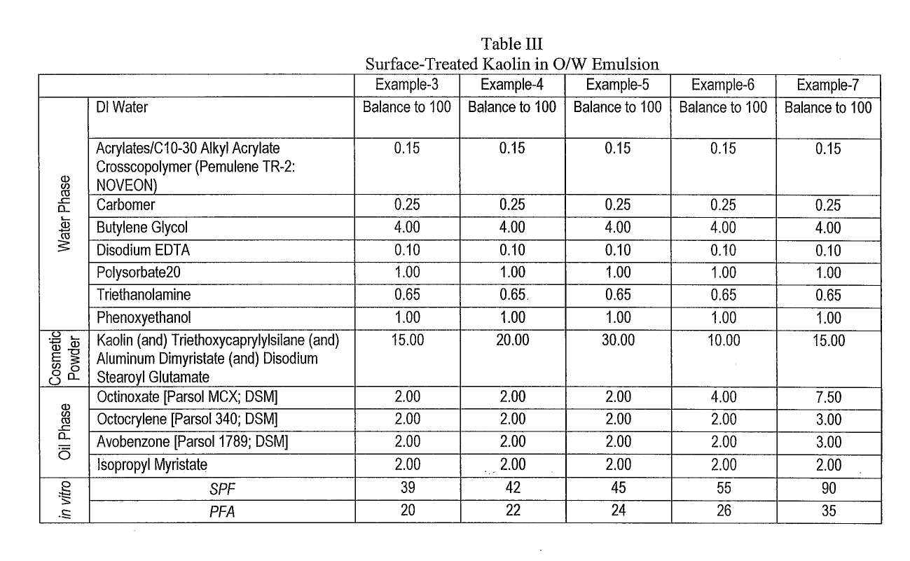

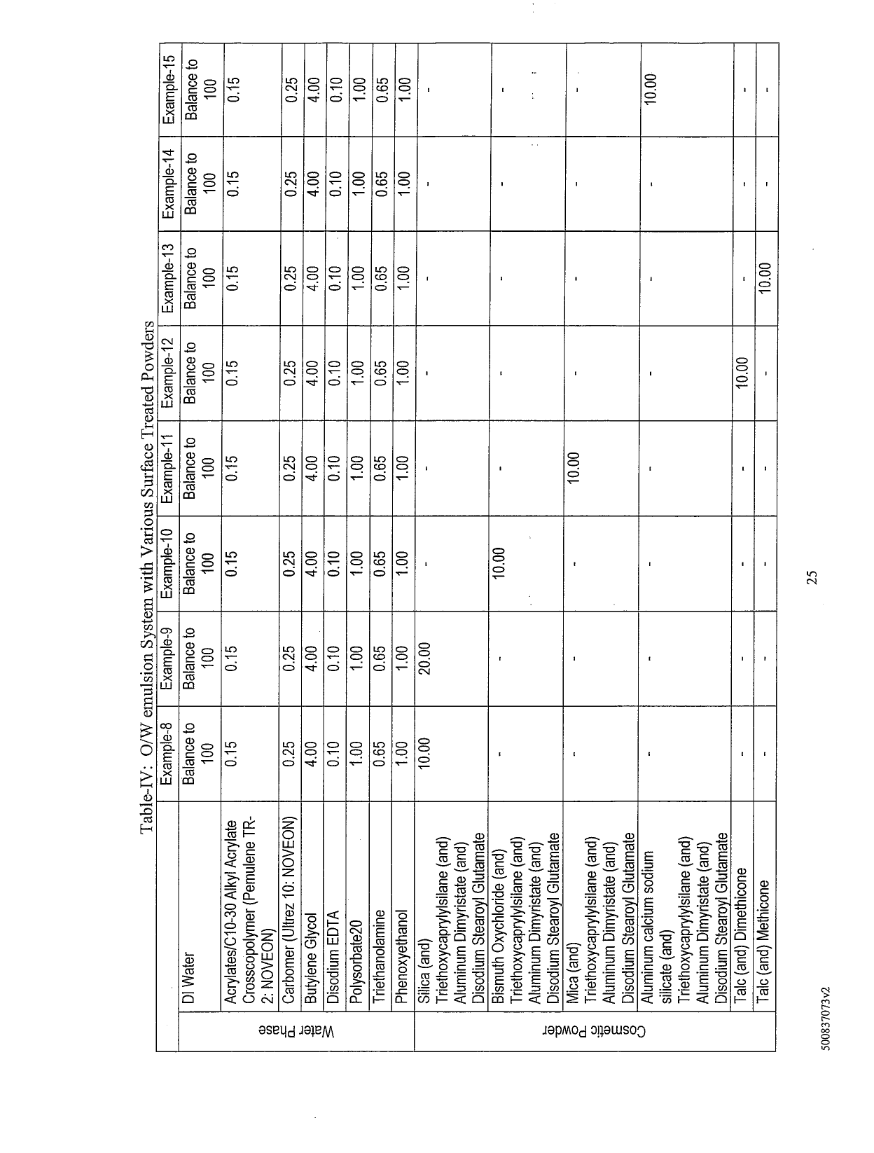

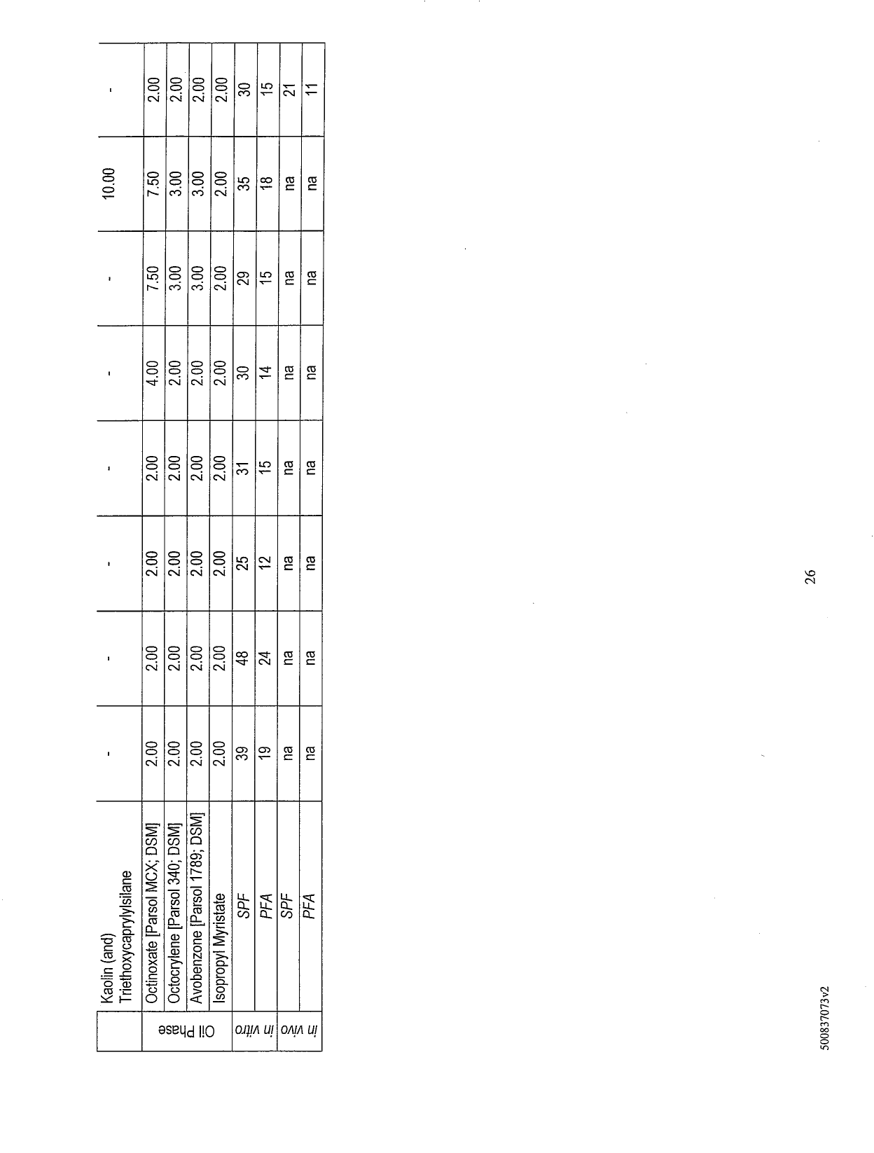

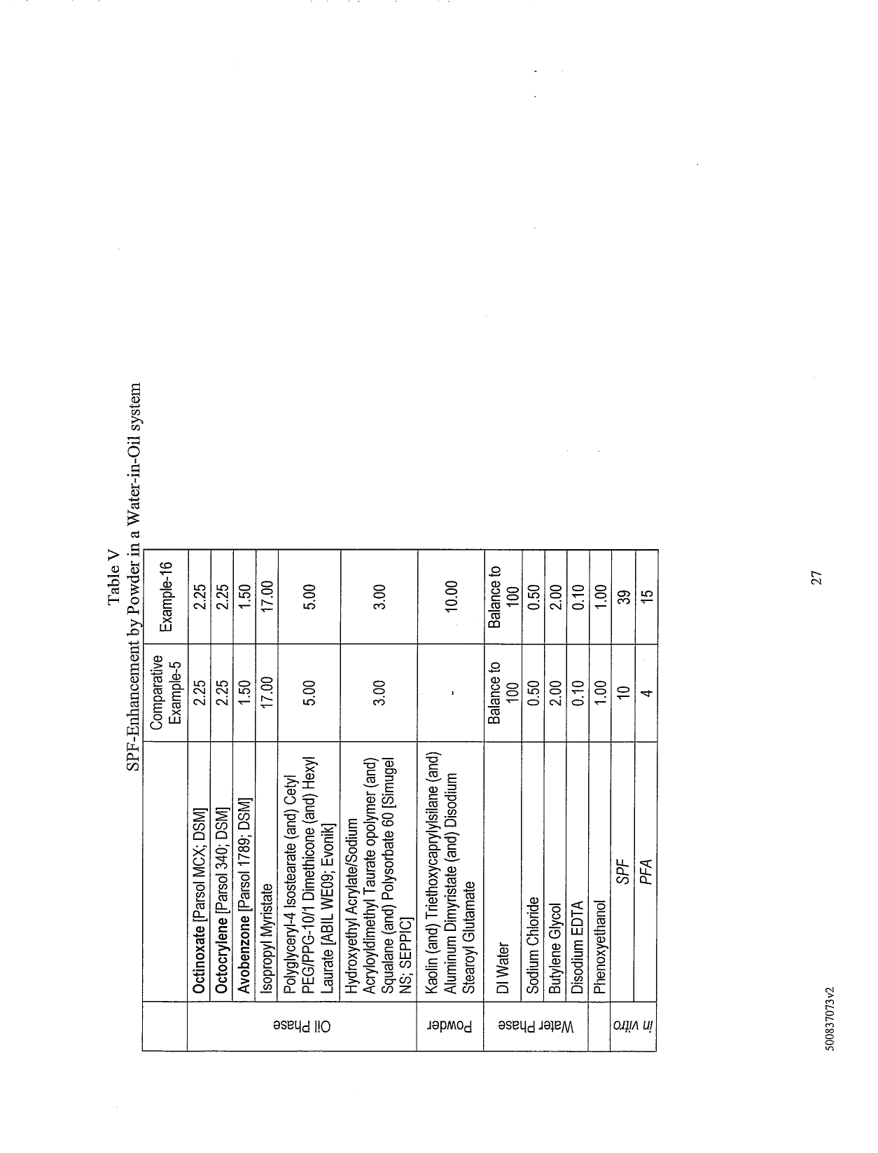

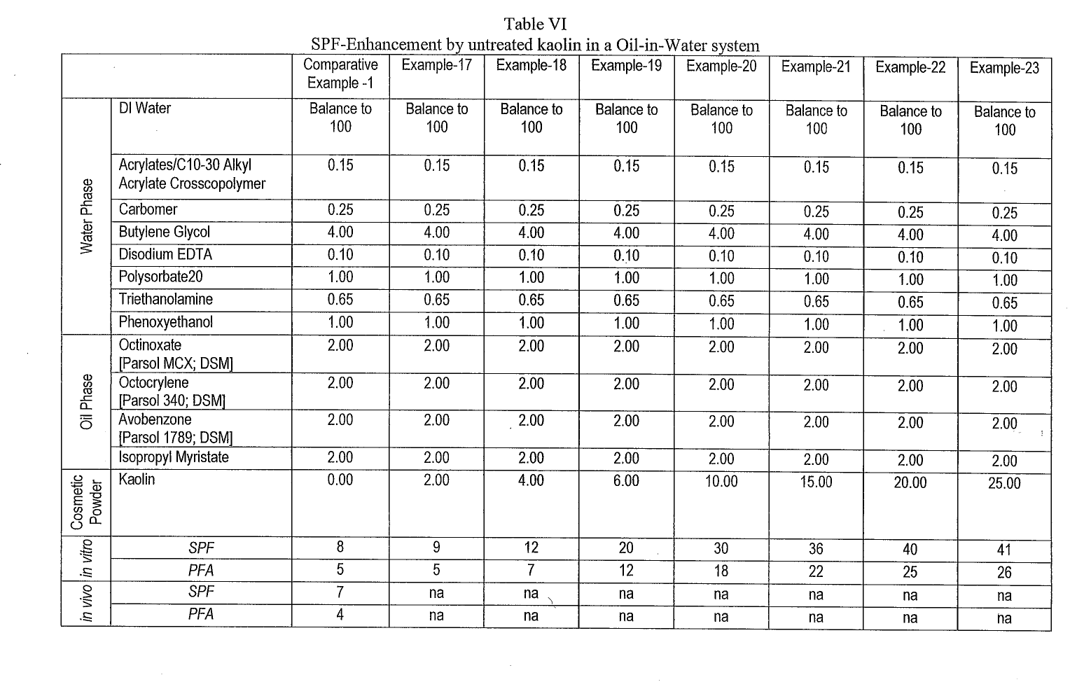

The ability of cosmetic powder materials to provide an SPF-enhancement is not limited to surface-treated powders. Table VI shows the effect of increasing concentrations of untreated kaolin on the SPF of an O/W emulsion similar to the one described in Table II. FIGS. 4 (SPF) and 5 (PFA) demonstrate that untreated kaolin increases the SPF of O/W systems. Moreover, the concentration dependence is sigmoid with an inflection point.

As shown in FIG. 1, the SPF observed for a given organic UV-active formulation depends in a sigmoid manner on the cosmetic powder concentration. The greatest concentration dependence is observed in the vicinity of the inflection point. FIG. 1 further shows that for a given cosmetic powder the position of the inflection depends on the composition of the organic UV-active. At concentrations above the inflection concentration, the SPF rises until a saturation (“plateau”) concentration is reached.

An aspect of the disclosure provides a desired SPF in the presence of lower organic UV-active concentrations by including at least one cosmetic powder at a concentration about or above an inflection concentration.

FIG. 1 further shows that at concentrations above the inflection concentration, the SPF rises until a saturation concentration is reached. Moreover, the saturation concentration for a given cosmetic powder depends on the nature of the organic UV-active. An aspect of the disclosure includes at least one cosmetic powder included at a concentration up to approximately a saturation concentration.

FIG. 1 demonstrates that surface-treated kaolin potentiates the SPF activity of the organic UV-active, but is not, itself, the UV-active agent. If UV-activity were a property of the treated kaolin, the titration curves in the presence of Banana Boat®, Coppertone®, and Neutrogena® should each be the same. However, the curves differ greatly. Thus showing that the treated kaolin is differentially potentiating the effect of the various UV-actives mixtures represented by these three commercial formulations.

TABLE I
Exemplary Formulation

| | | | Comparative |
|---|---|---|---|
| | | Example | Example |
| OIL | Octinoxate (PARSOL OMC)*1 | 2.00 | 2.00 |
| Phase | Octocrylene (PARSOL 340)*1 | 2.00 | 2.00 |
| | Avobenzone (PARSOL 1789)*1 | 2.00 | 2.00 |
| | Isopropyl Myristate (IPM) | 2.00 | 2.00 |
| WATER | DI Water | 70.00 | 80.00 |
| Phase | Acrylates/C10-30 Alkyl Acrylate | 0.15 | 0.15 |
| | Crosscopolymer (Pemulen TR-1) | | |
| | Carbomer (Ultrez 10) | 0.25 | 0.25 |
| | Butylene Glycol | 4.00 | 4.00 |
| | Na₂ EDTA | 0.10 | 0.10 |
| | TWEEN 20 | 0.50 | 0.50 |
| | Kaolin (and) Triethoxycaprylylsilane | 10.00 | — |
| | (and) Aluminum Dimyristate | | |
| | (and) Disodium Stearoyl Glutamate | | |
| | 18% NaOH aq. soln. | 1.00 | 1.00 |
| | Phenoxyethanol | 1.00 | 1.00 |
| | Acrylates Copolymer (Avalure AC118) | 5.00 | 5.00 |

| | | |
|---|---|---|
| Total | 100.00 | 100.00 |
| in vitro SPF | 35 ± 6 | 12 ± 2 |
| in vitro PFA | 21.6 | 8.3 |
| in vitro UVA/UVB | 0.773 | 0.769 |
| in vitro SPF Index | 5.8 | 2 |

Table I presents a non-limiting, exemplary formulation. The water and oil phases were formulated separately and then mixed.

An aqueous phase was formulated by loading deionized water into a high shear disperser mixer with acrylates (Pemulen® TR-1), carbomer (Ultrez® 10), and disodium EDTA. The mixture was blended at ˜1400 rpm with heating to ˜75° C. Upon dissolution of the solid materials, butylene glycol and Tween® 20 were added. The aqueous phase was completed by the dispersion of surface-treated kaolin.

An oil phase was separately formulated by heating the Octinoxate, octocrylene, avobenzone, and isopropyl myristate to ˜75° C.

The oil phase was added to the aqueous phase and emulsified by blending at ˜1400 rpm. The mixing blade was then changed from a disper to a propeller mixer. A sodium hydroxide solution was added to thicken the emulsion. Finally, phenoxyethanol and the acrylic copolymer (Avalure® AC118) were blended into the emulsion which was allowed to cool to room temperature with continued mixing.

The comparative example is an otherwise identical formulation with an additional portion of water in place of the treated kaolin. The formulation according to the invention yielded approximately three times the SPF of the comparative example.

This invention has industrial applicability in providing cosmetic formulations that provide high levels of SPF protection with low levels of organic, UV-actives.

The foregoing description of the invention illustrates and describes the present invention. Additionally, the disclosure shows and describes only the preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

Throughout this application, various references including publications, patents, and pre-grant patent application publications are referred to. Disclosures of these publications in their entireties are hereby incorporated by reference into this application to more fully describe the state of the art to which this invention pertains. It is specifically not admitted that any such reference constitutes prior art against the present application or against any claims thereof. All publications, patents, and pre-grant patent application publications cited in this specification are herein incorporated by reference, and for any and all purposes, as if each individual publication or patent application were specifically and individually indicated to be incorporated by reference. In the case of inconsistencies the present disclosure will prevail.

1. An SPF-boosting composition comprising:
up to 30 wt % of a non-volatile oil,
at least 30 wt % of an aqueous phase; and
about an SPF-inflection concentration of a cosmetic powder.

2. The SPF-boosting composition of claim 1, wherein said non-volatile oil comprises a UV-protective agent.

3. The SPF-boosting composition of claim 2, wherein said UV-protective agent comprises at least one non-volatile oil selected from the group consisting of octinoxate, octocrylene, avobenzone, PABA, octyldimethyl-PABA, Phenylbenzimidazole sulfonic acid, Cinoxate, Dioxybenzone (Benzophenone-8), Oxybenzone (Benzophenone-3), Homosalate, Menthyl anthranilate, Octisalate, Sulisobenzone, Trolamine salicylate, Terephthalylidene Dicamphor Sulfonic Acid, 4-Methylbenzylidene camphor, Methylene Bis-Benzotriazolyl Tetramethylbutylphenol, Bis-ethylhexyloxyphenol methoxyphenol triazine, bisimidazylate, Drometrizole Trisiloxane, Octyl triazone, Diethylamino Hydroxybenzoyl Hexyl Benzoate, Iscotrizinol, Polysilicone-15, Amiloxate, Ethylhexyl Dimethoxybenzylidene Dioxoimidazolidine Propionate, and mixtures thereof.

4. The SPF-boosting composition of claim 1, wherein said non-volatile oil further comprises an ancillary selected from the group consisting of oils and waxes.

5. The SPF-boosting composition of claim 4, wherein said ancillary oil is selected from the group consisting of isopropyl myristate, isotridecyl isononanoate, isostearyl isostearate, isocetyl isostearate, isopropyl isostearate, isodecyl isononanoate, cetyl octanoate, isononyl isononanoate, isopropyl myristate, isocetyl myristate, isotridecyl myristate, isostearyl palmitate, isocetyl palmitate, isodecyl palmitate, isopropyl palmitate, octyl palmitate, caprylic/capric triglyceride, glyceryl tri-2-ethylhexanoate, neopentyl glycol di(2-ethylhexanoate), diisopropyl dimerate, tocopherol, tocopherol acetate, avocado oil, camellia oil, turtle oil, macadamia nut oil, corn oil, mink oil, olive oil, grapeseed oil, egg yolk oil, sesame oil, wheat germ oil, castor oil, linseed oil, safflower oil, cotton seed oil, soybean oil, peanut oil, tea seed oil, rice bran oil, rice germ oil, jojoba oil, glycerol trioctanoate, glycerol triisopalmitate, trimethylolpropane triisostearate, pentaerythritol tetra-2-ethylhexanoate, lanolin, liquid lanolin, liquid paraffin, squalane, squalane, vaseline, and mixtures thereof.

6. The SPF-boosting composition of claim 4, wherein said ancillary wax is selected from the group consisting of paraffin wax, microcrystalline wax, ozokerite wax, ceresin wax, carnauba wax, candelilla wax, eicosanyl behenate, and mixtures thereof.

7. The SPF-boosting composition of claim 4, wherein said ancillary is a silicon oil selected from the group consisting of polymethylphenylsiloxane, polydiphenylsiloxane, polydiethylsiloxane, polydimethylsiloxane (dimethicone) more than 10 cSt and mixtures thereof.

8. The SPF-boosting composition of claim 1, wherein said cosmetic powder comprises a powder selected from the group consisting of organic pigments, inorganic pigments, pigment extenders, inorganic beads, organic beads, metal oxide powders, plastics, fillers for plastics, dehydrated dairy products, titanium dioxides, zinc oxides, zirconium dioxides, iron oxides, ultramarines, talc, chromium oxides, magnesium silicate, aluminum silicate, carbon black, cellulose, urethane, styrene, polyolefin, polyetheylene, polyamide, acrylates, calcium carbonate, zeolite, fumed silica, metal powders, ceramic powders, zirconium, modified starch, pearl, cotton powder, aluminum/magnesium silicate beads, silica beads, nylon beads, and combinations thereof.

9. The SPF-boosting composition of claim 8, wherein said cosmetic powder is coated with at least one surface-active agent chemically-immobilized to a surface thereof.

10. The SPF-boosting composition of claim 1, wherein said cosmetic powder is present at above an SPF-Index inflection concentration.

11. The SPF-boosting composition of claim 1, wherein said cosmetic powder is present at from about an SPF-Index inflection concentration to about an SPF-plateau concentration.

12. The SPF-boosting composition of claim 1, wherein said composition has an SPF-index of at least 3.0. of at least 4.0.

13. The SPF-boosting composition of claim 1, wherein said composition has an SPF-index of at least 3.0. of at least 5.0.

14. The SPF-boosting composition of claim 1, wherein said composition is free of volatile silicone oil.

15. The SPF-boosting composition of claim 1, comprising from about 5 wt % to about 30 wt % of a cosmetic powder.

16. The SPF-boosting composition of claim 13, comprising from about 10 wt % to about 25 wt % of a cosmetic powder.

17. The SPF-boosting composition of claim 9, wherein said cosmetic powder is coated with a plurality of surface-active agents chemically-immobilized to a surface thereof.

18. The SPF-boosting composition of claim 17, wherein said plurality of surface-active agents comprises triethoxycaprylylsilane, aluminum dimyristate, and disodium stearoyl glutamate.
